(19) United States
(12) Patent Application Publication
(10) Pub. No.: US 2023/0141079 A1
(43) Pub. Date: May 11, 2023

(54) Methods, Systems, and Devices for Facilitating a Health Protection Protocol

(71) Applicant: Soonbum Shin, Laguna Woods, CA (US)

(72) Inventor: Soonbum Shin, Laguna Woods, CA (US)

(21) Appl. No.: 17/522,755

(22) Filed: Nov. 9, 2021

(51) Int. Cl. G16H 40/67; G16H 10/65; G16H 15/00; G16H 40/20; G16H 50/70; G06F 16/901; A61B 5/00; A61B 5/0205; A61B 5/024; A61B 5/11; A61B 5/145

(52) U.S. Cl. CPC G16H 40/67; G16H 10/65; G16H 15/00; G16H 40/20; G16H 50/70; G06F 16/9024; A61B 5/7475; A61B 5/746; A61B 5/747; A61B 5/7405; A61B 5/02055; A61B 5/02405; A61B 5/11; A61B 5/14542; A61B 5/4809

(57) ABSTRACT

A method for facilitating a health protection protocol is presented. The present invention provides the storage device, the processing device, the health tracking device, and the at least one user interface device, where the health tracking device contains at least one sensor, at least one user biological data is received through the at least one sensor. At least one user biological data is transmitted from the health tracking device to the at least one user interface device. The at least one user biological data is transmitted from the at least one user interface device to the storage device. The plurality of health data is updated with the at least one user biological data through the processing device. The plurality of health data is transferred to the at least one user interface device. An at least one health statistic is generated from the plurality of health data through the processing device.

The current application claims a priority to the U.S. Provisional Patent application Ser. No. 63/198,730 filed on Nov. 9, 2020.

### FIELD OF THE INVENTION

The present invention relates generally to a field of data processing. More specifically, the present invention is methods, systems, and devices for facilitating a health protection protocol.

### BACKGROUND OF THE INVENTION

In recent times, there has been increase in outbreaks in the form of epidemics and pandemics which have affected millions of people worldwide. For example, the recent Coronavirus disease 2019 (COVID-19) has a global death-to-case ratio (which reflects the number of deaths divided by the number of diagnosed cases within a given time interval) as 2.6% (1,207,284/46,937,042) as of 3 Nov. 2020, according to recent statistics. Also, mortality rates associated with the COVID-19 are highly correlated to age. In those younger than 50 years, the risk of death is less than 0.5%, while in those older than 70 it is more than 8%. According to Centers for Disease Control and Prevention (CDC) analysis, the risk of death by age groups in the United States is 0.003%, 0.02%; 0.5% and 5.4% for the age groups 0-19, 20-49, 50-69, and 70 or over, respectively. Researchers have continuously been devising various methodologies to curb the effects resulting from such outbreaks. An individual affected with such an outbreak generally develops a rise in the body temperature as a first symptom, and an early detection of any such symptom may help curbing the spread of the outbreak. People who have been made to stay away from their families owing to the outbreaks may want to take care of the elderly members of their families, which are at a greater risk to catch the infections than most of the other age groups.

Existing techniques for facilitating a health protection protocol are deficient with regard to several aspects. For instance, current technologies do not provide trends for various parameters such as, temperature, heart rate, etc., which can be used as a certificate for confirming the well-being of an individual of the past few days at various instances. Furthermore, current technologies do not facilitate for providing medical assistance in an emergency situation instantly by gestures. Moreover, current technologies do not facilitate combination of periodic monitoring of the various parameters of members in a community in real-time by an administrator who can take necessary actions as and when required.

Therefore, there is a need for improved methods, systems, and devices for facilitating a health protection protocol that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope. According to some embodiments, a method to facilitate a health protection protocol, in accordance with some embodiments. Further, the health protection protocol may be referred to as AnyCARE health protection (AHP) system. Further, the health protection protocol may include a health tracking device (may be referred to as AnyCARE TAP health tracking device), a software application, and a data visualization interface (such as, a at least one user interface device). Further, the health tracking device, in an instance, may monitor one or more parameters of a body of a user. Further, the user, in an instance, may wear the health tracking device on a wrist. Further, the software application (may be referred to as a user application) may be downloaded on a user device. Further, a software application programming interface (or software API), in an instance, may display at least one trend corresponding to each parameter of the one or more parameters on the user device. Further, the at least one trend, in an instance, may include current trends and historical trends. Further, a second software application (may be referred to as a family application) may be downloaded on the user device. Further, the data visualization interface may display the at least one trend on at least one device associated with at least one health protection protocol custodian.

Accordingly, the method may include a step of generating, using at least one sensor, a temperature reading of the user on the health tracking device. Further, the method may include a step of receiving, using an at least one user interface device, the temperature reading of the user on the user device. Further, the method may include a step of retrieving, using a storage device, the one or more temperature readings of the user. Further, the method may include a step of analyzing, using a processing device, the one or more temperature readings of the user. Further, the method may include a step of determining, using the processing device, at least one temperature trend based on the analyzing.

Further, a custom firmware may be implemented on the health tracking device configured for performing one or more tasks related to the health tracking device. Further, the health tracking device, in an instance, may display one or more second details corresponding to the user based on the custom firmware implemented on the health tracking device. Further, the custom firmware, in an instance, may configured to display a certificate of one or more days based on the determining of the at least one trend using the processing device.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

FIG. 1 is an illustration of an online platform consistent with various embodiments of the present disclosure.

FIG. 2 is a block diagram representation of a system to facilitate a health protection protocol, in accordance with some embodiments.

FIG. 3 is a portion view of an exemplary representation of a system structure associated with the health protection protocol, in accordance with some embodiments.

FIG. 4 is a portion view of the exemplary representation of the system structure associated with the health protection protocol, in accordance with some embodiments.

FIG. 5 is a block diagram of a computing device for implementing the methods disclosed herein, in accordance with some embodiments.

FIG. 6 is a flowchart of the present invention that shows a health monitoring protocol.

FIG. 7 is a continuing flowchart of the present invention taken along continuing arrow 1 in FIG. 1.

FIG. 8 is a flowchart of the present invention that shows a daily status report being generated from an at least one health statistic.

FIG. 9 is a flowchart of the present invention that shows worker input being tasked by an administrator through an assignment interface.

FIG. 10 is a flowchart of the present invention that shows a messaging interface.

FIG. 11 is a flowchart of the present invention that shows an at least one historical data being generated from the plurality of health data.

FIG. 12 is a flowchart of the present invention that shows an emergency alert feature.

FIG. 13 is a flowchart of the present invention that shows a locating feature used in conjunction with the emergency alert feature.

FIG. 14 is a flowchart of the present invention that shows a disconnection notification feature.

FIG. 15 is a flowchart of the present invention that shows a device finder feature.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein. Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of facilitating a health protection protocol, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, and at least one sensor. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include an at least one user interface device configured for communicating with one or more external devices. The one or more external devices may include, for example, but are not limited to, a client device, a third-party database, public database, a private database and so on. Further, the at least one user interface device may be configured for communicating with the one or more external devices over one or more communication channels. Further, the one or more communication channels may include a wireless communication channel and/or a wired communication channel. Accordingly, the at least one user interface device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices in order to perform authentication. For example, the one or more steps may include receiving, using the at least one user interface device, the secret human readable data from an input device such as, for example, a keyboard, a keypad, a touch-screen, a microphone, a camera and so on. Likewise, the one or more steps may include receiving, using the at least one user interface device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more-time gaps between performance of any two steps of the one or more steps.

Further, in some embodiments, the one or more predefined conditions may be specified by the one or more users. Accordingly, the one or more steps may include receiving, using the at least one user interface device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

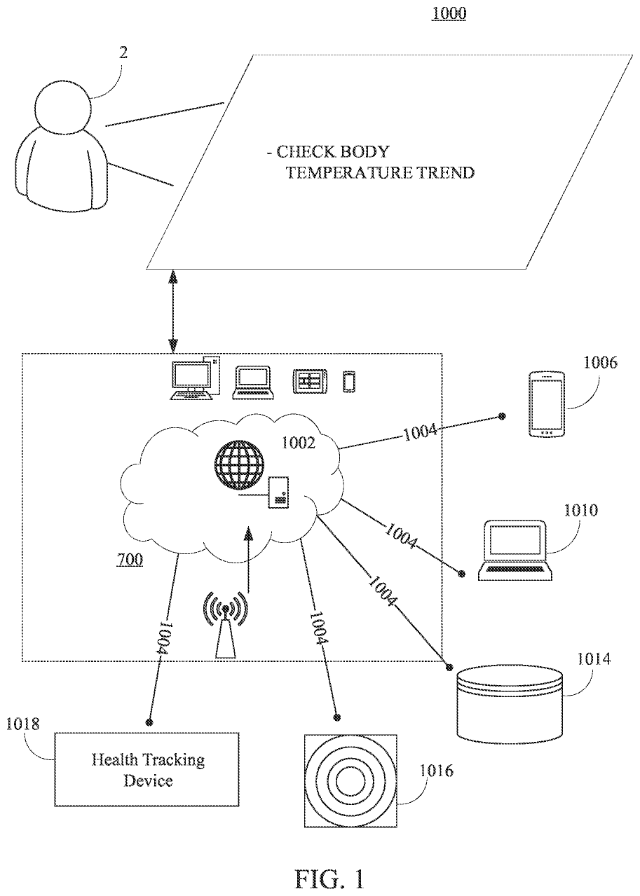

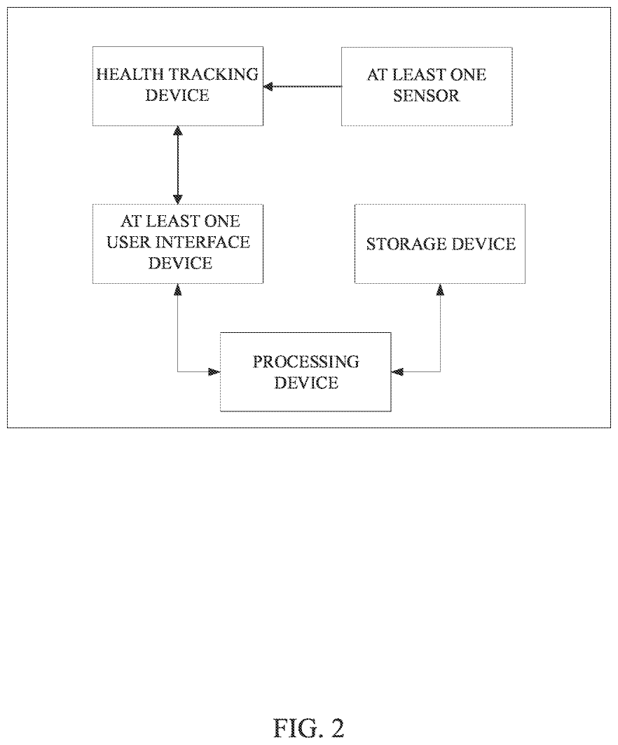

In reference to FIGS. 1-2, the present disclosure aims to provide a safe and healthy work or living environment under coronavirus pandemic. Further, the present invention describes a protocol (may be referred to as AnyCARE Health Protection (AHP) system) which aims to contain the spread of COVID-19 in the community by monitoring any fever among community members and screening out a potential COVID-19 infection early. The fever is the first symptom of COVID-19 for most patients with symptoms. Further, the protocol disclosed in the disclosure consists of a health tracking device, the at least one user interface device for the user and family, and a at least one user interface device for the caregiver to monitor the health status of their community members in real-time. The community can be a senior living facility, cruise ship, school, hotel or any workplace.

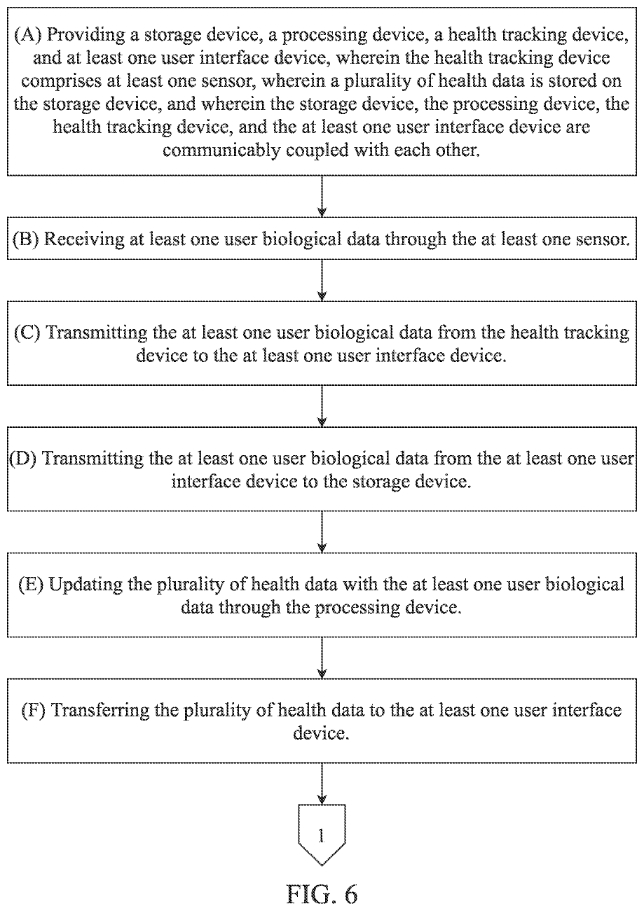

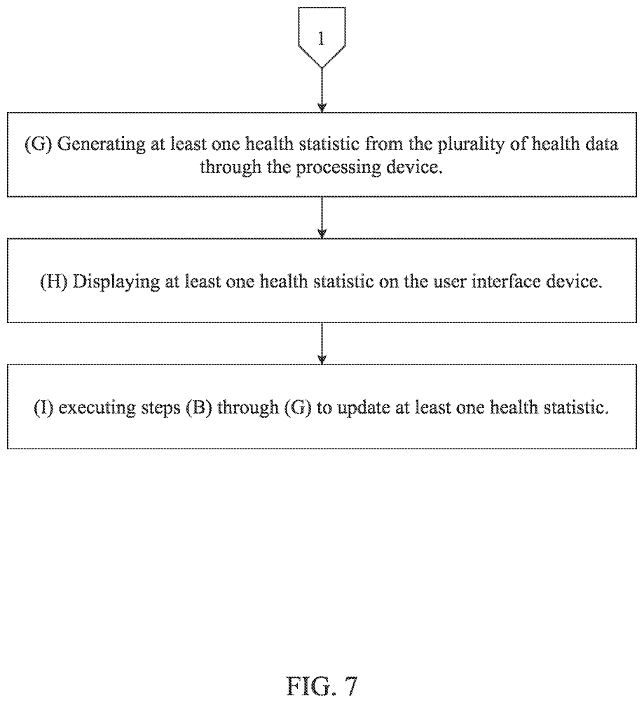

In reference to FIGS. 6-7, the present invention provides the storage device, the processing device, the health tracking device, and the at least one user interface device, where the health tracking device comprises at least one sensor, where a plurality of health data is stored on the storage device, and where the storage device, the processing device, the health tracking device, and the at least one user interface device are communicably coupled with each other (Step A). At least one user biological data is received through the at least one sensor (Step B). The at least one user biological data is transmitted from the health tracking device to the at least one user interface device (Step C). The at least one user biological data is transmitted from the at least one user interface device to the storage device (Step D). The plurality of health data is updated with the at least one user biological data through the processing device (Step E). The plurality of health data is transferred to the at least one user interface device (Step F). An at least one health statistic is generated from the plurality of health data through the processing device (Step G). The at least one health statistic is displayed on the user interface device (Step H). Finally, steps (B) through (G) are executed again to update at least one health statistic (Step I).

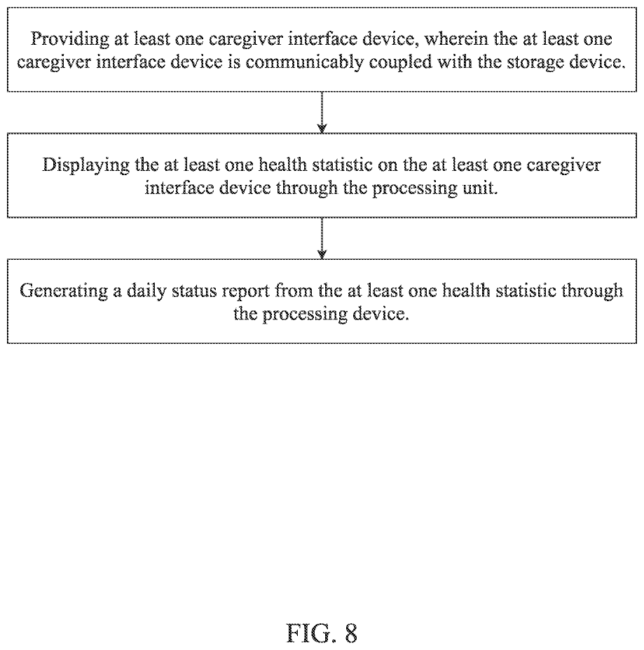

In reference to FIG. 8, at least one caregiver interface device is provided, where the at least one caregiver interface device is communicably coupled with the storage device. The at least one health statistic is displayed on the at least one caregiver interface device through the processing unit. A daily status report is generated from the at least one health statistic through the processing device. The present invention features caregiver monitoring functions. In addition to monitoring functions, the present invention also allows caregiver's to generate daily status reports that compile data derived from the at least one health statistic.

The least one sensor of the health tracking device comprises a temperature sensor, heartbeat sensor, a step sensor, a blood oxygen sensor, a heart rate variability sensor, and any other suitable sensor that will facilitate in receiving at least one biological reading. The at least one user biological data comprises a user temperature reading, where the user temperature reading is received through the temperature sensor. The at least one user biological data further comprises a user heartbeat reading where the user heartbeat reading is received through the heartbeat sensor. The at least one user biological data further comprises a user step reading where the user step reading is received through the step sensor. The at least one user biological data further comprises a user blood oxygen reading where the user blood reading is received through the blood oxygen sensor. The at least one user biological data further comprises a heart rate variability reading. The user heart rate variability reading is received through the heart rate variability sensor. In the preferred embodiment of the present invention, the at least one user biological data further comprises a sleep status reading where the user sleep status reading is received through the at least one sensor suitable for detecting the sleep status of the user.

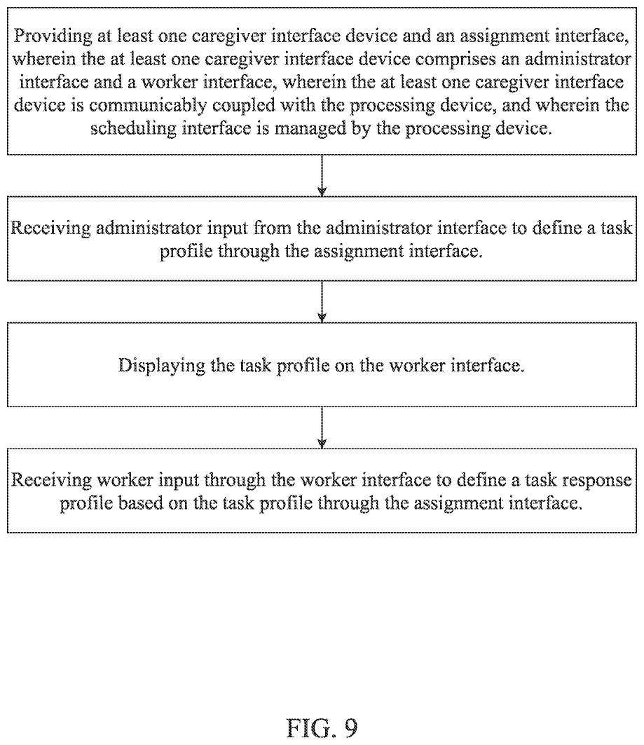

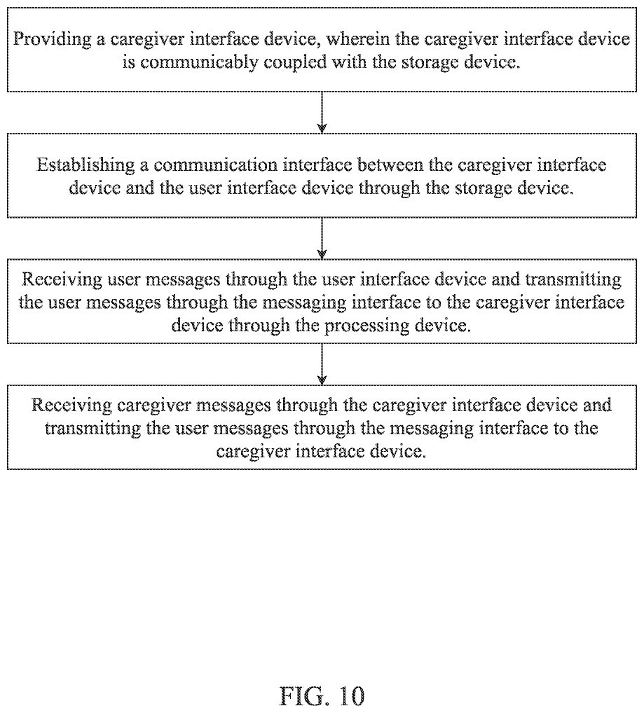

In reference to FIGS. 9-10. the present invention provides at least one caregiver interface device and an assignment interface where the at least one caregiver interface device comprises an administrator interface and a worker interface. The at least one caregiver interface device is communicably coupled with the processing device where the scheduling interface is managed by the processing device. Administrator input is received from the administrator interface to define a task profile through the assignment interface. The task profile is displayed on the worker interface. Worker input is received through the worker interface to define a task response profile based on the task profile through the assignment interface. The at least one caregiver interface device is communicably coupled with the storage device. A communication interface is established between the caregiver interface device and the user interface device through the storage device. User messages are received through the user interface device and transmitting the user messages through the messaging interface to the caregiver interface device through the processing device. Caregiver messages are received through the caregiver interface device. The user messages are transmitted through the messaging interface to the caregiver interface device.

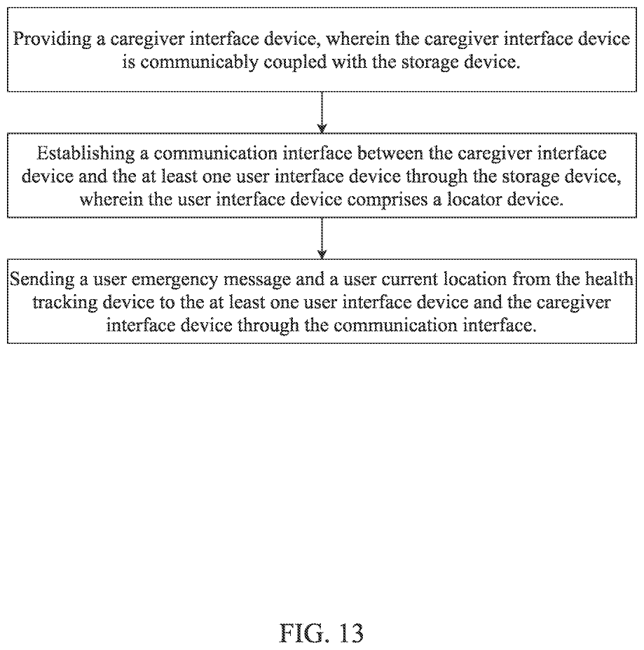

In reference to FIG. 13, the user interface device comprises a locator device. A user emergency message and a user current location is sent from the health tracking device to the at least one user interface device and the caregiver interface device through the communication interface.

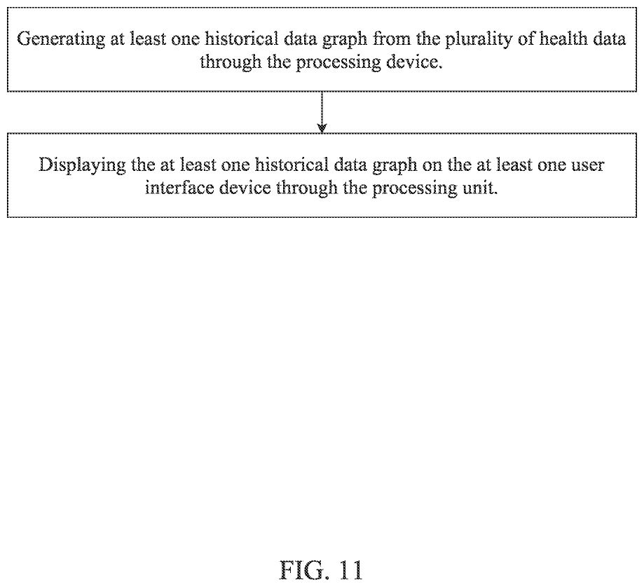

In reference to FIG. 11, an at least one historical data graph is generated from the plurality of health data through the processing device. The at least one historical data graph is displayed on the at least one user interface device through the processing unit.

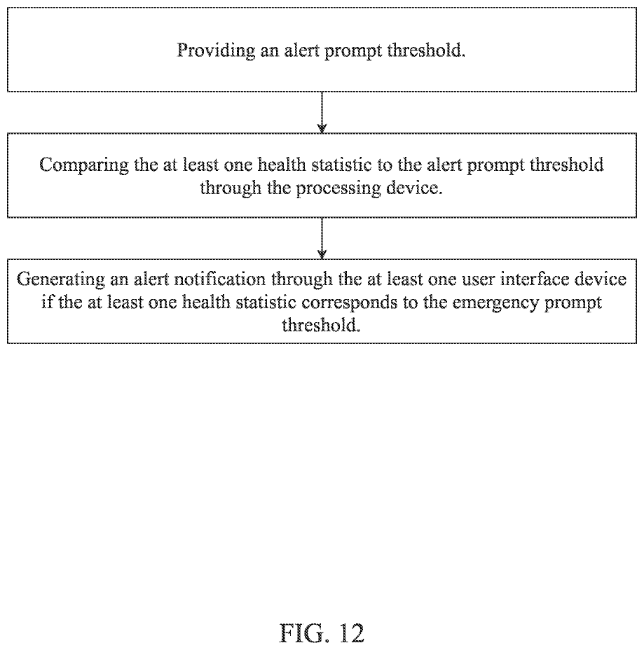

In reference to FIG. 12, the present invention provides an alert prompt threshold. The at least one health statistic is compared to the alert prompt threshold through the processing device. An alert notification is generated through the at least one user interface device if the at least one health statistic corresponds to the emergency prompt threshold.

The health tracking device disclosed in the disclosure is a unique smartwatch innovation, which combines fitness tracker, thermometer, pulse oximeter and medical alert function into one package. The health tracking device monitors the user's body temperature every 30 minutes and store the data for 30 days. It has two temperature screens, one for daily trend and another for two weeks of historical trend data, which can be used as a temperature certificate to replace two weeks quarantine. Further, the smartphone application disclosed in the disclosure can share the current and historical trends of temperature, blood oxygen level, heart rate, exercise level and sleep patterns among user, family and caregivers. Further, the at least one user interface device disclosed in the disclosure displays the health data of all community members and receives a warning message in case of any high body temperature incident detected by the health tracking device. The caregiver can send alert messages from the at least one user interface device to the community member's health tracking device(s) in case of any incident.

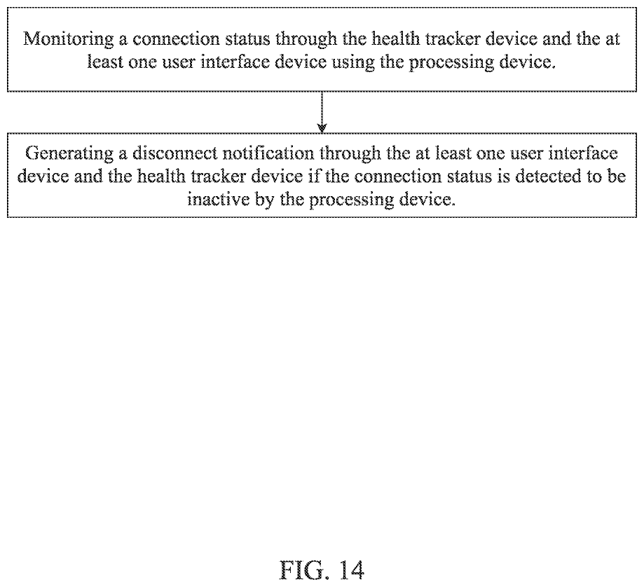

In reference to FIG. 14, a connection status is monitored through the health tracking device and the at least one user interface device using the processing device. A disconnect notification is generated through the at least one user interface device and the health tracking device if the connection status is detected to be inactive by the processing device.

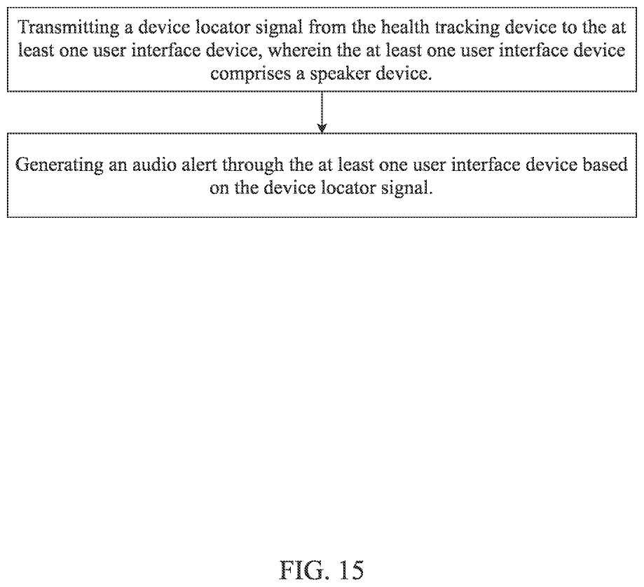

In reference to FIG. 15, a device locator signal is transmitted from the health tracking device to the at least one user interface device where the at least one user interface device comprises a speaker device. An audio alert is generated through the at least one user interface device based on the device locator signal.

Further, the present disclosure aims for early detection of any fever symptom among community members, as fever is the first sign of COVID-19 infected people showing symptoms. The early detection can be a very effective way of preventing a possible spread of coronavirus in the community. Further, the present disclosure aims to empower each individual member to manage health with the 24/7 body temperature information and refrain from moving around in case of any fever to minimize further exposure to other people. Further, the present disclosure aims to provide a safe and healthy community environment with actively monitoring the health status of their community members and share the information with family and caregiver. Further, the present disclosure aims to provide an affordable, but reliable safety tool for senior living facilities and cruise liners to manage the safety of their guests and workers proactively. The health tracking device include features such as but not limited to body temperature trend display on the health tracking device, daily and weekly up to 4 weeks, which can be used as a verification tool for free of fever for any gate or access control, owner' ID on the watch; name, phone number and MAC address of the health tracking device to prove the temperature data on the watch belong to the wearer of the health tracking device, make the health tracking device vibrate when the health tracking device loses wireless connection pairing with the phone. The user can send an SOS message by double tapping the SOS screen to the family and caregiver. The phone can also be enabled to receive the incoming call-in speaker mode when the user can't get to the phone nearby. Vital signs are collected for 30 days in the health tracking device. Further, specifications of the health tracking device may include Bluetooth 5.0, Display: 1.3″, at least one sensor comprising PPG, Accelerometer, Temperature, and Heart rate sensors, Battery lifetime: 7 days, IP67 water resistant, Data saving for 30 days, and a woven Nylon band. Further, the key features of the health tracking device include automatic Monitoring and Display of at least one biological reading such as but not limited to Body temperature, Steps, Heart Rate, Sleep Status, and two-week readings of body temperature history. Additionally, the health tracking device may further feature Manual Measurement functions for Heart Rate, Blood Oxygen (SpO2), Heart Rate Variability (HRV), and Temperature. Furthermore, the health tracking device may feature notification prompts such as but not limited Incoming call, Incoming message, and the disconnect notification,

In the caregiver interface device, the caregiver can open the user's account and configure the individual user's health tracking device and app remotely. Furthermore, the caregiver can send messages from the at least one user interface device to the user's health tracking device screen. A master caregiver can assign regional caregivers to manage the local members in the region using the administrator interface. The at least one user interface device can select application mode, user mode or family mode. Furthermore, the application can create and send approval codes for the family and caregiver to view the user's health data. Under the family mode, the user can view the health status of the family members. SMS messages can be sent to the family and caregiver with the user's location information. Furthermore, the present invention may be implemented for the senior living facilities. The health tracking device monitors various parameters such as body temperature, heart rate, SpO2, HRV, activity level, and display various reminders including medication of all residents and workers. The at least one user interface device displays the current and historical trends of temperature, blood oxygen level, heart rate, exercise level, sleep patterns, etc. The storage device, such as a cloud server stores the current and historical trends of temperature, blood oxygen level, heart rate, exercise level, sleep patterns, etc. A daily temperature alert status report may be sent to the safety officer on a caregiver's every morning, and the report will contain the list of residents and workers showing fever symptoms along with an overnight temperature history.

Further, the present invention may be implemented for the cruise liners. The health tracking device is provided to each passenger 2-3 weeks before a boarding date such that the health tracking device may monitor, in an instance, body temperature, heart rate, activity level, and display messages from a safety officer in a ship and successful pairing with a user's phone. Temperature data is tracked using the health tracking device during boarding, such as current body temperature and a daily minimum temperature history or a daily maximum temperature history during the last 2 weeks. The storage device stores the current and historical trends of temperature, blood oxygen level, heart rate, exercise level, sleep patterns, etc. A daily temperature alert status report may be sent to the ship safety officer on a 24/7 desktop at least one user interface device every morning, and the report will contain the list of residents and workers showing fever symptoms along with an overnight temperature history.

Further, the present invention may be implemented for school settings. Each student and teacher are provided with a health tracking device such that the health tracking device may monitor, in an instance, body temperature, heart rate, activity level, and display messages from the safety officer at the school and successful pairing with a user's phone. The health tracking device can send overnight body temperature data to the school every morning, such as a current body temperature and an overnight minimum temperature or an overnight maximum temperature. The storage device stores the current and historical trends of temperature, blood oxygen level, heart rate, exercise level, sleep patterns, etc. A temperature alert status report may be generated and sent to the safety officer at the school through the at least one user interface device every morning before 7 am during weekdays, and the report will contain the list of students showing fever symptoms along with their contact phone number and an overnight temperature history. Referring now to figures, FIG. 1 is an illustration of an online platform 1000 consistent with various embodiments of the present disclosure. By way of non-limiting example, the online platform 1000 to facilitate a health protection protocol may be hosted on a centralized server 1002, such as, for example, a cloud computing service. The centralized server 1002 may communicate with other network entities, such as, for example, a mobile device 1006 (such as a smartphone, a laptop, a tablet computer, etc.), other electronic devices 1010 (such as desktop computers, server computers, etc.), databases 1014, sensors 1016, actuators (not shown) and a health tracking device 1018 over a communication network 1004, such as, but not limited to, the Internet. Further, users of the online platform 1000 may include relevant parties such as, but not limited to, end-users, administrators service providers, service consumers and so on. Accordingly, in some instances, electronic devices operated by the one or more relevant parties may be in communication with the online platform 1000.

A user 1012, such as the one or more relevant parties, may access the online platform 1000 through a web-based software application or browser. The web-based software application may be embodied as, for example, but not be limited to, a website, a web application, a desktop application, and a mobile application compatible with a computing device 700.

FIG. 2 is a block diagram representation of a system to facilitate a health protection protocol, in accordance with some embodiments. Further, the system may include at least one sensor, a processing device, an at least one user interface device, and a storage device.

Further, the at least one sensor (such as, sensors 1016) may be communicatively coupled to the at least one user interface device. Further, the at least one sensor, in an instance, may be any sensor that may be disposed of in a health tracking device associated with the health protection protocol. Further, the at least one sensor may include sensors such as, but are not limited to, a temperature sensor, a heart rate sensor, a photoplethysmography (PPG) sensor, etc. Further, the at least one sensor may be configured to sense one or more parameters of a body of a user. Further, at least one reading associated with the one or more parameters may be generated subsequent to the sensing of the one or more parameters by the at least one sensor.

Further, the at least one user interface device may be communicatively coupled with the processing device. Further, the at least one user interface device may be configured for receiving the at least one reading associated with the one or more parameters on a user device (such as, mobile device 1006 or other electronic devices 1010). Further, the user device may include devices such as, but are not limited to, smartphones, laptops, PCs, etc. Further, the at least one user interface device may be configured to establish a communication link between the user device and the health tracking device. Further, the user device may receive one or more temperature readings over the communication link from the health tracking device. Further, the at least one user interface device may be configured for transmitting at least one trend corresponding to the one or more parameters to at least one health protection protocol custodian. Further, the at least one user interface device may be configured to establish a communication link between the user device and at least on device associated with the at least one health protection protocol custodian. Further, the at least one device may receive the at least one temperature trend of the user over the communication link. Further, the at least one user interface device may be configured for transmitting at least one alert on the health tracking device and/or the user device from the at least one device.

Further, the processing device may be configured for analyzing one or more temperature readings of the user. Further, the processing device may be configured for determining the at least one trend associated with the one or more parameters. Further, the processing device may be configured to generate a report based on the at least one trend.

Further, the storage device (such as, a centralized server 1002) may be communicatively coupled with the processing device. Further, the storage device may be configured to store the one or more temperature readings based on the receiving of the one or more temperature readings from the user device.

FIGS. 6-7 is flowchart of a method to facilitate a health protection protocol, in accordance with some embodiments. Further, the health protection protocol may be referred to as AnyCARE health protection (AHP) system. Further, the health protection protocol may include the health tracking device (may be referred to as AnyCARE TAP health tracking device), a software application, and a data visualization interface (such as, a at least one user interface device). Further, the health tracking device, in an instance, may monitor one or more parameters of a body of a user. Further, the one or more parameters may include parameters such as, but are not limited to, temperature, blood oxygen, heart rate, heart rate variability (HRV), activity level, etc. Further, the user, in an instance, may wear the health tracking device on a wrist. Further, the software application (may be referred to as a user application) may be downloaded on a user device. Further, the user device may include devices such as, but are not limited to, smartphones, laptops, PCs, etc. Further, a software application programming interface (or software API), in an instance, may display at least one trend corresponding to each parameter of the one or more parameters on the user device. Further, the at least one trend, in an instance, may include current trends and historical trends. Further, the current trends may include at least one first current health data of each parameter associated with the body of the user (for example, if the parameter is temperature, then the at least one first current health data may include a minimum temperature and a maximum temperature in a current duration of time). Further, the historical trends may include at least one first past health data of each parameter associated with the body of the user (for example, if the parameter is temperature, then the at least one first past health data may include a minimum temperature and a maximum temperature in a past duration of time). Further, the software application, in an instance, may provide a platform for sharing of the at least one trend with one or more second users, one or more members of a family of the user, and at least one health protection protocol custodian. Further, a second software application (may be referred to as a family application) may be downloaded on the user device. Further, the data visualization interface may display the at least one trend on at least one device (such as, mobile device 1006 or other electronic devices 1010) associated with the at least one health protection protocol custodian. Further, the at least one device may include devices such as, but are not limited to, smartphones, laptops, PCs, etc. Further, the data visualization interface, in an instance, may display the at least one first current health data and the at least one first past health data. Further, at least one community of members may be monitored using the health protection protocol by the at least one health protection protocol custodian. Further, the second software API may facilitate the monitoring of the at least one community of members. Further, the at least one community of members may correspond to places such as, but are not limited to, senior living facilities, cruise ships, schools, hotels, etc.

Accordingly, the method may include a step of generating, using at least one sensor, a temperature reading of the user on the health tracking device. Further, the at least one sensor, in an instance, may be any sensor that may be disposed of in the health tracking device. Further, the at least one sensor may be configured to sense a temperature of the body of the user. Further, the temperature reading may be generated subsequent to the sensing of the temperature by the at least one sensor. Further, the health tracking device, in an instance, may monitor the temperature of the body of the user at least once every thirty minutes subsequent to the generating. Further, the health tracking device, in an instance, may be configured to store the at least one first past health data for at least thirty days based on the monitoring.

Further, the method may include a step of receiving, using a at least one user interface device, the temperature reading of the user on the user device. Further, a communication link may be established between the user device and the health tracking device. Further, the user device may receive one or more temperature readings over the communication link from the health tracking device. Further, the software API may be configured for displaying the one or more temperature readings of the user on the user device based on the receiving. Further, the software API may facilitate switching between at least two modes. Further, the at least two modes, in an instance, may include a user mode and a family mode. Further, the user, in an instance, may establish the communication link with one or more health tracking devices using the software API on the user device. Further, the establishing, in an instance, may be based on at least one approval code for each health tracking device of the one or more health tracking devices. Further, the at least one approval code may facilitate successful authentication of each health tracking device for establishing the communication link with the user device. Further, each health tracking device may be associated with each community member of the at least one community of members (such as, members of the family of the user). Further, the user may monitor the one or more temperature readings of each community member. Further, the second software API, in an instance, may facilitate the monitoring of the one or more temperature readings of each community member.

Further, the method may include a step of retrieving, using the storage device, the one or more temperature readings of the user. Further, the storage device, in an instance, may correspond to a cloud server. Further, the storage device may establish the communication link with the user device. Further, the storage device may be configured to store the one or more temperature readings based on receiving of the one or more temperature readings from the user device. Further, the storage device, in an instance, may be configured to store the at least one first current health data and the at least one first past health data. Further, the storage device may establish the communication link with the at least one device of the at least one health protection protocol custodian. Further, the at least one health protection protocol custodian may monitor the one or more temperature readings, the at least one first current health data, and the at least one first past current health data as and when needed. Further, the one or more temperature readings, in an instance, may include the temperature readings of the at least one community of members.

Further, the method may include a step of analyzing, using a processing device, the one or more temperature readings of the user. Further, the one or more temperature readings, in an instance, may include the temperature readings of the at least one community of members.

Further, the method may include a step of determining, using the processing device, at least one temperature trend based on the analyzing. Further, the at least one temperature trend may be based on the one or more temperature readings of the user. Further, the at least one temperature trend, in an instance, may be based on the at least one first past health data associated with the user. Further, the at least one temperature trend, in an instance, may include one or more details associated with the temperature of the body of the user based on the at least one first past health data and the at least one first current health data. Further, the one or more details may include a daily average temperature, an average of the maximum temperature, an average of the minimum temperature, etc. Further, the at least one temperature trend, in an instance, may include temperature trends of the at least one community of members. Further, in an instance, the at least one temperature may be displayed on the health tracking device subsequent to the determining. Further, the determining, in an instance, may display the at least one temperature trend on the user device based on receiving of the at least one temperature trend over the communication link.

Further, the method may include a step of transmitting, using the at least one user interface device, the at least one temperature trend to the at least one health protection protocol custodian. Further, the at least one device associated with the at least one health protection protocol custodian may receive the at least one temperature trend of the user based on the transmitting over the communication link. Further, the data visualization interface, in an instance, may display one or more temperature trends on the at least one device. Further, each temperature trend of the one or more temperature trends, in an instance, may be associated with each community member of the at least one community of members. Further, the at least one health protection protocol custodian, in an instance, may take an action based on the at least one temperature trend. Further, the taking of the action, in an instance, may include transmitting at least one alert on the health tracking device and/or the user device associated with the user. Further, the transmitting, in an instance, may include sending of the at least one alert to each health tracking device associated with each community member. Further, in some embodiments, the at least one alert may include a message in a form of a text on the user device with varying degrees of color and/or sound that may intensify based on a severity of the at least one alert.

FIGS. 12-13 is a set of flowcharts of a method to facilitate a health protection protocol in case of an emergency, in accordance with some embodiments. Accordingly, the method may include a step of generating, using a processing device, the alert notification or the emergency message using a health tracking device. Further, the alert notification may be generated based on an interaction of a user with the health tracking device in an emergency situation such as, but is not limited to, fall down, extreme heart rate, low temperature, etc. Further, the interaction, in an instance, may include tapping on a display of the health tracking device. Further, at least of the at least one sensor (such as, an accelerometer) may facilitate sensing of the interaction on the health tracking device. Further, the alert notification, in an instance, may be generated based on the sensing. Further, the method may include a step of receiving, using the at least one user interface device, the alert notification on the at least one user interface device. Further, the receiving, in an instance, may be based over a communication link established between the user device and the health tracking device. Further, in some embodiments, the user device may receive a plurality of alert notifications from each community member of at least one community of members (such as, members of a family of the user). Further, the plurality of alert notifications may be assigned a hierarchical order based on a severity of each alert notification of the plurality of alert notifications.

Further, the method may include a step of transmitting, using the at least one user interface device, the alert notification on at least one device associated with at least one health protection protocol custodian. Further, the transmitting, in an instance, may be based over a communication link established between the user device and the at least one device. Further, the plurality of alert notifications may be received on the at least one device based on the transmitting. Further, the at least one health protection protocol custodian may determine a severity associated with the emergency situation. Further, the at least one health protection protocol custodian may take an action based on the determining. Further, each alert notification of the plurality of alert notifications may be considered for taking an action by the at least one health protection protocol custodian in the hierarchical order based on the severity. Further, in an instance, the at least one health protection protocol custodian may dial a contact number associated with an emergency service for providing medical aid to the user for overcoming the emergency situation.

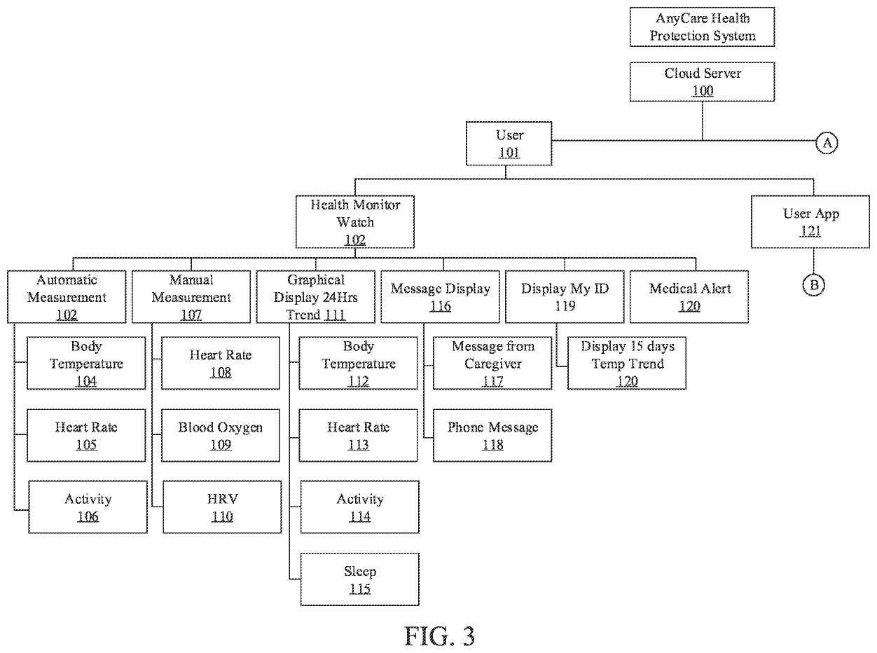

FIG. 3 is a portion view of an exemplary representation of a system structure associated with a health protection protocol, in accordance with some embodiments. Further, a cloud server, in an instance, may be configured to store at least one first current health data and at least one first past health data. Further, the cloud server, in an instance, may facilitate a real-time database configured for monitoring of the at least one first current health data of a user in real-time on a user device using a software application and/or on a health tracking device. Further, the user, in an instance, may monitor the at least one first current health data and/or the at least one first past health data of each community member of at least one community of members.

Further, the health tracking device may display a graphical user interface (GUI) inclusive of at least one trend based on an implementation of a custom firmware on the health tracking device. Further, the GUI, in an instance, may display one or more parameters (such as, body temperature, heart rate, etc.) associated with a body of the user on the health tracking device. Further, the at least one trend may be displayed for at least twenty-four hours based on monitoring of the one or more parameters at a regular interval using the health tracking device. Further, one or more alerts may be received on the health tracking device from at least one health protection protocol custodian. Further, the one or more alerts, in an instance, may include textual information and/or graphical information that the user may implement in order to keep the one or more parameters within normal limits.

Further, the custom firmware may be configured for displaying one or more second details corresponding to the user. Further, the one or more second details may include details such as, but are not limited to, a name of the user, a phone number associated with the user, a media access control (MAC) address, etc. Further, the custom firmware, in an instance, may configured to display a temperature report of one or more days based on the determining of the at least one trend (such as, a temperature trend). Further, the temperature report, in an instance, may be used as a report confirming a well-being state of the user.

Further, the health tracking device may facilitate generating of a medical alert based on an interaction of the user with the health tracking device in an emergency situation. Further, the interaction, in an instance, may include tapping on the health tracking device. Further, at least of the at least one sensor (such as, an accelerometer) may facilitate sensing of the interaction on the health tracking device. Further, the medical alert, in an instance, may be generated based on the sensing.

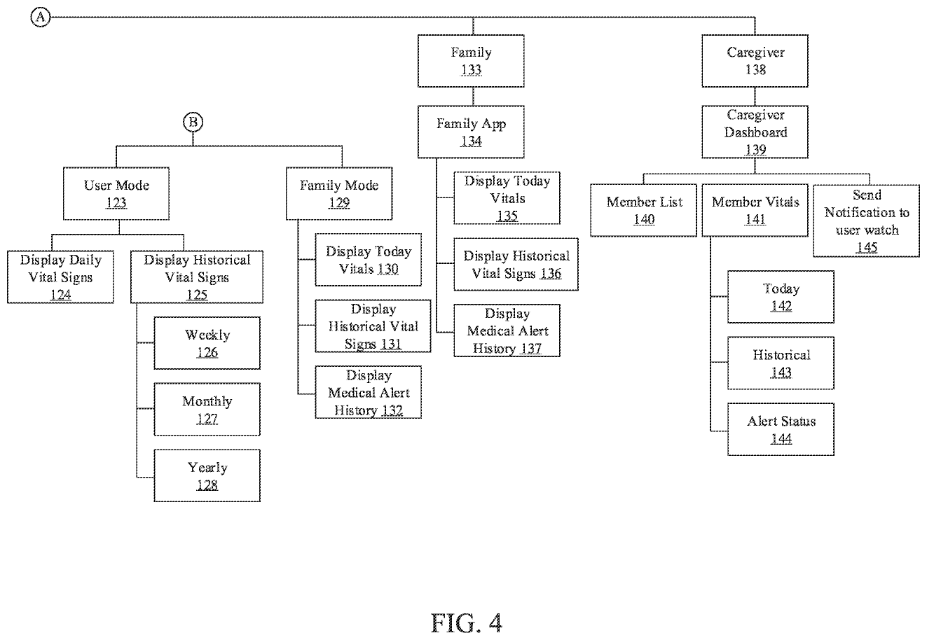

FIG. 4 is a portion view of an exemplary representation of a system structure associated with a health protection protocol, in accordance with some embodiments (explained in conjunction with FIG. 3). Further, a software API associated with the health protection protocol may facilitate switching between at least two modes based on an interaction with a user device associated with a user. Further, the at least two modes, in an instance, may include a user mode and a family mode.

Further, in the user mode, the software API may be configured to display current statistics of the one or more parameters based on monitoring of the one or more parameters by the health tracking device. Further, the displaying, in an instance, may include at least one daily vital sign associated with the one or more parameters. Further, in the user mode, the software API may be configured to display past statistics of the one or more parameters based on the monitoring of the one or more parameters by the health tracking device. Further, the displaying, in an instance, may include a report based on a duration of time. Further, the report based on the duration of time may include a weekly report, a monthly report, and/or a yearly report.

Further, in the family mode, the software API may be configured to display statistics of the one or more parameters associated with each community member of at least one community of members based on the monitoring of the one or more parameters by each health tracking device of one or more health tracking devices associated with each community member. Further, the displaying, in an instance, may include at least one daily vital sign, at least one historical vital sign, and at least one medical alert history.

Further, a second software API associated with the health protection protocol may be configured to display statistics of the one or more parameters associated with each community member of at least one community of members based on the monitoring of the one or more parameters by each health tracking device of one or more health tracking devices associated with each community member. Further, the displaying, in an instance, may include at least one daily vital sign, at least one historical vital sign, and at least one medical alert history.

Further, at least one device associated with at least one health protection protocol custodian send one or more notifications to at least one community of members. Further, the one or more notifications, in an instance, may include alerts that the user may implement in order to keep the one or more parameters associated with each community member of the at least community of members within normal limits.

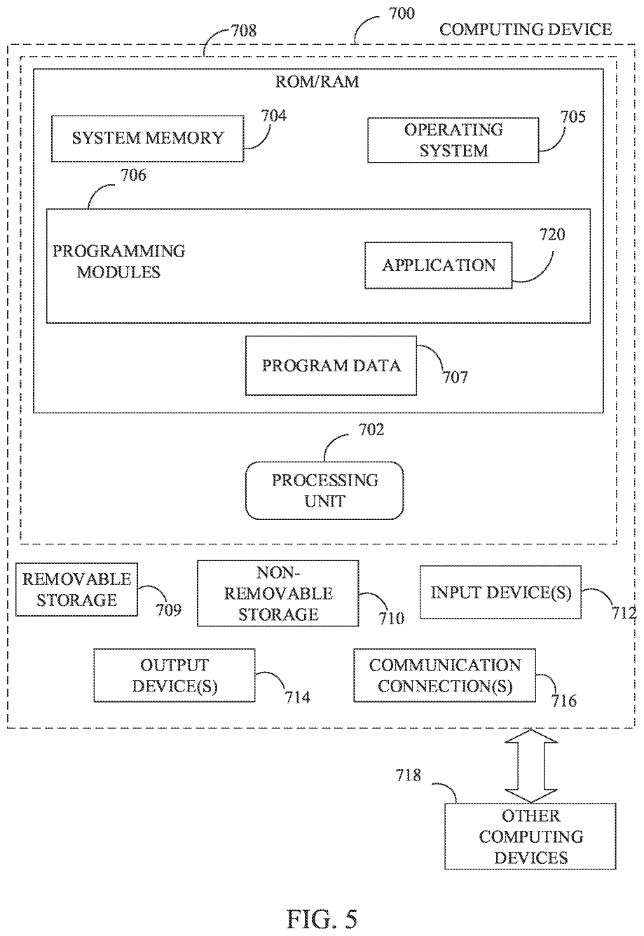

With reference to FIG. 5, a system consistent with an embodiment of the disclosure may include a computing device or cloud service, such as computing device 700. In a basic configuration, computing device 700 may include at least one processing unit 702 and a system memory 704. Depending on the configuration and type of computing device, system memory 704 may comprise, but is not limited to, volatile (e.g.

random-access memory (RAM)), non-volatile (e.g. read-only memory (ROM)), flash memory, or any combination. System memory 704 may include operating system 705, one or more programming modules 706 (such as, at least one sensor module, a communication protocol module, etc.), and may include a program data 707. Operating system 705, for example, may be suitable for controlling computing device 700's operation. Furthermore, embodiments of the disclosure may be practiced in conjunction with a graphics library, other operating systems, or any other application program and is not limited to any particular application or system. This basic configuration is illustrated in FIG. 5 by those components within a dashed line 708.

Computing device 700 may have additional features or functionality. For example, computing device 700 may also include additional data storage devices (removable and/or non-removable) such as, for example, magnetic disks, optical disks, or tape. Such additional storage is illustrated in FIG. 7 by a removable storage 709 and a non-removable storage 710. Computer storage media may include volatile and nonvolatile, removable and non-removable media implemented in any method or technology for storage of information, such as computer-readable instructions, data structures, program modules, or other data. System memory 704, removable storage 709, and non-removable storage 710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information, and which can be accessed by computing device 700. Any such computer storage media may be part of device 700. Computing device 700 may also have input device(s) 712 such as a keyboard, a mouse, a pen, a sound input device, a touch input device, a location sensor, a camera, a biometric sensor, etc. Output device(s) 714 such as a display, speakers, a printer, etc. may also be included. The aforementioned devices are examples and others may be used. Computing device 700 may also contain a communication connection 716 that may allow device 700 to communicate with other computing devices 718, such as over a network in a distributed computing environment, for example, an intranet or the Internet.

Communication connection 716 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 704, including operating system 705. While executing on processing unit 702, programming modules 706 (e.g., application 720 such as a media player) may perform processes including, for example, one or more stages of methods, algorithms, systems, applications, servers, databases as described above. The aforementioned process is an example, and processing unit 702 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention as hereinafter claimed.

What is claimed is:

1. A method for facilitating a health protection protocol comprising:
(A) providing a storage device, a processing device, a health tracking device, and at least one user interface device, wherein the health tracking device comprises at least one sensor, wherein a plurality of health data is stored on the storage device, and wherein the storage device, the processing device, the health tracking device, and the at least one user interface device are communicably coupled with each other;
(B) receiving at least one user biological data through the at least one sensor;
(C) transmitting the at least one user biological data from the health tracking device to the at least one user interface device;
(D) transmitting the at least one user biological data from the at least one user interface device to the storage device;
(E) updating the plurality of health data with the at least one user biological data through the processing device;
(F) transferring the plurality of health data to the at least one user interface device;
(G) generating at least one health statistic from the plurality of health data through the processing device;
(H) displaying at least one health statistic on the user interface device; and
(I) executing steps (b) through (g) to update at least one health statistic.

2. The method for facilitating a health protection protocol as claimed in claim 1 comprising:
providing at least one caregiver interface device, wherein the at least one caregiver interface device is communicably coupled with the storage device;
displaying the at least one health statistic on the at least one caregiver interface device through the processing unit; and
generating a daily status report from the at least one health statistic through the processing device.

3. The method for facilitating a health protection protocol as claimed in claim 1 comprising:
providing at least one caregiver interface device and an assignment interface, wherein the at least one caregiver interface device comprises an administrator interface and a worker interface, wherein the at least one caregiver interface device is communicably coupled with the processing device, and wherein the scheduling interface is managed by the processing device;
receiving administrator input from the administrator interface to define a task profile through the assignment interface;
displaying the task profile on the worker interface; and
receiving worker input through the worker interface to define a task response profile based on the task profile through the assignment interface.

4. The method for facilitating a health protection protocol as claimed in claim 1 comprising:
providing a caregiver interface device, wherein the caregiver interface device is communicably coupled with the storage device;
establishing a communication interface between the caregiver interface device and the user interface device through the storage device;
receiving user messages through the user interface device and transmitting the user messages through the messaging interface to the caregiver interface device through the processing device; and
receiving caregiver messages through the caregiver interface device and transmitting the user messages through the messaging interface to the caregiver interface device.

5. The method for facilitating a health protection protocol as claimed in claim 1 comprising:
generating at least one historical data graph from the plurality of health data through the processing device; and
displaying the at least one historical data graph on the at least one user interface device through the processing unit.

6. The method for facilitating a health protection protocol as claimed in claim 1 comprising:
providing an alert prompt threshold;
comparing the at least one health statistic to the alert prompt threshold through the processing device; and
generating an alert notification through the at least one user interface device if the at least one health statistic corresponds to the emergency prompt threshold.

7. The method for facilitating a health protection protocol as claimed in claim 6 comprising:
providing a caregiver interface device, wherein the caregiver interface device is communicably coupled with the storage device;
establishing a communication interface between the caregiver interface device and the at least one user interface device through the storage device, wherein the user interface device comprises a locator device;
sending a user emergency message and a user current location from the health tracking device to the at least one user interface device and the caregiver interface device through the communication interface.

8. The method for facilitating a health protection protocol as claimed in claim 1 comprising:
monitoring a connection status through the health tracking device and the at least one user interface device using the processing device; and
generating a disconnect notification through the at least one user interface device and the health tracking device if the connection status is detected to be inactive by the processing device.

9. The method for facilitating a health protection protocol as claimed in claim 1 comprising:
transmitting a device locator signal from the health tracking device to the at least one user interface device, wherein the at least one user interface device comprises a speaker device; and
generating an audio alert through the at least one user interface device based on the device locator signal.

10. The method for facilitating a health protection protocol as claimed in claim 1, wherein the at least one sensor comprises a temperature sensor, wherein the at least one user biological data comprises a user temperature reading, and wherein the user temperature reading is received through the temperature sensor.

11. The method for facilitating a health protection protocol as claimed in claim 1, wherein the at least one sensor comprises a heartbeat sensor, wherein the at least one user biological data comprises a user heartbeat reading, and wherein the user heartbeat reading is received through the heartbeat sensor.

12. The method for facilitating a health protection protocol as claimed in claim 1, wherein the at least one sensor comprises a step sensor, wherein the at least one user biological data comprises a user step reading, and wherein the user step reading is received through the step sensor.

13. The method for facilitating a health protection protocol as claimed in claim 1, wherein the at least one sensor comprises a blood oxygen sensor, wherein the at least one user biological data comprises a user blood oxygen reading, and wherein the user blood reading is received through the blood oxygen sensor.

14. The method for facilitating a health protection protocol as claimed in claim 1, wherein the at least one sensor comprises a heart rate variability sensor, wherein the at least one user biological data comprises a heart rate variability reading, and wherein the user heart rate variability reading is received through the heart rate variability sensor.

15. The method for facilitating a health protection protocol as claimed in claim 1, wherein the at least one user biological data comprises a sleep status reading and wherein the user sleep status reading is received through the at least one sensor.

16. A method for facilitating a health protection protocol comprising:
(A) providing a storage device, a processing device, a health tracking device, a caregiver interface device, and at least one user interface device, wherein the health tracking device comprises at least one sensor, wherein a plurality of health data is stored on the storage device, wherein the caregiver interface device is communicably coupled with the storage device, and wherein the storage device, the processing device, the health tracking device, and the at least one user interface device are communicably coupled with each other;
(B) receiving at least one user biological data through the at least one sensor;
(C) transmitting the at least one user biological data from the health tracking device to the at least one user interface device;
(D) transmitting the at least one user biological data from the at least one user interface device to the storage device;
(E) updating the plurality of health data with the at least one user biological data through the processing device;
(F) transferring the plurality of health data to the at least one user interface device;
(G) generating at least one health statistic from the plurality of health data through the processing device;
(H) displaying at least one health statistic on the user interface device;
(I) executing steps (b) through (g) to update at least one health statistic;
(J) establishing a communication interface between the caregiver interface device and the user interface device through the storage device;
(K) receiving user messages through the user interface device and transmitting the user messages through the messaging interface to the caregiver interface device through the processing device; and
(L) receiving caregiver messages through the caregiver interface device and transmitting the user messages through the messaging interface to the caregiver interface device.
